(19) United States
(12) Patent Application Publication
(10) Pub. No.: US 2008/0033021 A1
(43) Pub. Date: Feb. 7, 2008

(54) WASH COMPOSITION FOR THE HANDLING OF CONTACT LENSES

(76) Inventor: Susan Burke, Batavia, NY (US)

Correspondence Address: Bausch & Lomb Incorporated, One Bausch & Lomb Place, Rochester, NY, 14604-2701, US

(21) Appl. No.: 11/768,209

(22) Filed: Jun. 26, 2007

Related U.S. Application Data
(60) Provisional application No. 60/835,017, filed on Aug. 2, 2006.
(60) Provisional application No. 60/835,018, filed on Aug. 2, 2006.

(51) Int. Cl. A01N 37/52; A01N 25/00; A01N 43/40; A61P 31/00

(52) U.S. Cl. 514/358; 514/635; 514/772

(57) ABSTRACT

A method for minimizing microbial contamination of contact lenses during the handling of the lenses. The method comprises instructing a person that wears contact lenses to wash their hands prior to the handling of the contact lenses with a wash composition. The wash composition includes one or more surfactants selected from the group consisting of non-ionic, cationic, amphoteric or zwitterionic surfactants, and optionally, an anionic surfactant. The wash composition also includes an antimicrobial agent that is compatible with ocular tissue. The invention is also directed to a kit for to assist contact lens wearers with the care maintenance of their lenses. The kit includes the wash composition and a multipurpose contact lens solution. The wash composition is suitable for use with the multipurpose contact lens solution.

This application claims priority to U.S. provisional application 60/835,018, filed Aug. 8, 2006, and U.S. provisional application 60/835,017, filed Aug. 8, 2006 under 35 U.S.C. § 119(e)(1).

This invention relates to a wash composition that is compatible with the safe and effective care of contact lenses, and to a kit that includes the wash composition and a multipurpose contact lens solution.

### BACKGROUND OF THE INVENTION

Contact lenses are a convenient and effective way of correcting vision that has grown in popularity to over 30 million users worldwide. Some contact lenses, including extended wear contact lenses, require routine care including disinfection, cleaning and contact lens conditioning. Disinfection refers to the prevention of infection to the eye of a patient caused by microbes. Microbes are primarily introduced to the eye by the handling of a contact lens. Cleaning of a contact lens refers to removal of debris, protein or lipids from the surface of a contact lens. Conditioning of a contact lens refers to treating the contact lens with an agent that improves the comfort of the lens.

Multipurpose solutions such as ReNu® and ReNu® MultiPlus by Bausch & Lomb, Rochester, N.Y., are designed to clean, disinfect and condition contact lenses. A multipurpose solution is used in accordance with a required regimen. For example, ReNu MultiPlus requires a patient to rinse each side of their contact lenses and soak the contact lens for a minimum of four hours. Patients are also instructed to thoroughly wash their hands with soap just prior to handling the lenses and to use fresh solution each time they clean and disinfect their lenses. On some occasions components in the soap can be transferred to the contact lens or contact lens case during handling, e.g., rubbing, of the contact lenses. Many hand soaps, however, contain components, e.g., anionic surfactants, that can negatively impact the disinfectant properties of the multipurpose solution, and thereby, decrease its effectiveness against microorganisms. Thus, there is a need for a soap formulation or wash composition that excludes or at least minimizes the concentration of such components that can negatively impact the biocidal properties of a multipurpose lens solution. The present invention addresses this need.

### SUMMARY OF THE INVENTION

The invention is directed to a method for minimizing microbial contamination of contact lenses during the handling of the lenses. The method comprises instructing a person that wears contact lenses to wash their hands prior to the handling of the contact lenses with a wash composition. The wash composition includes one or more surfactants selected from the group consisting of non-ionic, cationic, amphoteric or zwitterionic surfactants, and optionally, an anionic surfactant. However, the anionic surfactant, if present, is present at a concentration that is 30 wt. % or less of the total surfactant concentration. The wash composition also includes an antimicrobial agent that is compatible with ocular tissue.

The invention is also directed to a kit to assist contact lens wearers with the care maintenance of their lenses. The kit comprises a wash composition that includes one or more surfactants selected from the group consisting of non-ionic, cationic, amphoteric or zwitterionic surfactants, and optionally, an anionic surfactant. Again, the anionic surfactant, if present, is present at a concentration that is 30 wt. % or less of the total surfactant concentration. The wash composition also includes an antimicrobial agent that is compatible with ocular tissue. The kit also comprises a multipurpose contact lens solution. The wash composition is suitable for use with the multipurpose contact lens solution such that any one component in the wash composition at a reduced concentration of 5% the concentration in the wash composition does not negatively impact the biocidal efficacy of the multipurpose solution.

### DETAILED DESCRIPTION OF THE INVENTION

It is understood by those in the contact lens industry that most contact lens wearers do not comply with the lens usage, storage and cleaning regimens and protocols recommended by contact lens and contact lens solution manufacturers. It is also understood that most contact lens wearers do not comply with the care and usage guidelines provided by their eye physician.

Evidence of non-compliance was derived from two independent studies conducted on behalf of Bausch & Lomb. The first study was an Internet panel-based survey of 2,369 contact lens wearers across seven European countries (UK, Germany, France, Italy, Spain, Netherlands and Sweden). This study was used to provide an overview on the compliance levels of daily disposable contact lens wearers. The second study reported on 1,402 wearers of two-weekly or monthly-replaced lenses aged 16-64 years across seven European countries (UK, Germany, France, Italy, Spain, Russia and Poland). The second study was an Internet survey of contact lens use and covered areas such as the duration and frequency of contact lens wear, methods of lens cleaning and disinfecting, the lens case and lens storage and the discussion of recommended care guidelines between the patient and their eye care professional.

The findings of the two studies demonstrated that greater than 80% of all contact lens wearers do not fully comply with the correct care regimen for the insertion and removal of contact lenses. For example, only one in four respondents (23%) claim to never wear their lenses beyond the recommended period. In particular, contact lens wearers with bi-weekly lenses are more likely to over-wear their lenses than those with monthly lenses. Also, younger patients are more likely to over-wear their contact lens wearers compared to older patients.

The findings from the two studies also demonstrated that contact lens wearers do not always wash their hands prior to inserting, removing or cleaning (rubbing) their lenses. For example, 28% of contact lens wearers do not wash their hands before inserting their lenses, and 43% of contact lens wearers do not wash their hands prior to removing them. Of those patients that do regularly wash their hands prior to handling their lenses fourteen (14) percent use only water for washing their hands prior to insertion of lenses and sixteen (16) percent prior to their removal.

Infections are most commonly suffered when microorganisms are transmitted directly to the contact lens by contaminated hands or surfaces such as a contaminated lens case. Accordingly, it is important to wash ones hands prior to the insertion and removal of contact lenses to minimize the risk of infection. It is also important to periodically wash the lens case.

Accordingly, this application describes a wash composition that contains ingredients that are compatible with most components common to contact lens disinfecting solutions presently on the market. Accordingly, the surfactant(s) of the wash composition is selected from the group consisting of non-ionic, cationic, amphoteric or zwitterionic surfactants to minimize any negative interaction that can exist between anionic surfactants and the cationic antimicrobial agents common to most, if not all, contact lens disinfecting solutions.

The invention is directed to a method for minimizing microbial contamination of contact lenses during the handling of the lenses. The method includes instructing a person that wears contact lenses to wash their hands prior to the handling of the contact lenses with a particular wash composition. This wash composition comprises one or more surfactants selected from the group consisting of non-ionic, cationic, amphoteric or zwitterionic surfactants, and an antimicrobial agent that is compatible with ocular tissue.

The total surfactant concentration in the composition will be from 5 wt. % to 40 wt. %. Typically, the amount of non-ionic, cationic, amphoteric or zwitterionic surfactant is a minimum of 10 wt. %, 12 wt. %, 15 wt. % or 18 wt. % and/or a maximum of 30 wt. % or 25 wt. %.

The composition may also include an anionic surfactant, however, the anionic surfactant, if present, is present at a concentration that is 30 wt. % or less of the total surfactant concentration in the composition. For example, if the total surfactant concentration in the composition is about 20 wt. %, then the anionic surfactant can be present at less than 6wt. % in the composition. In one instance, the anionic surfactant, if present, is present at a concentration that is 10 wt. % or less of the total surfactant concentration.

In many preferred embodiments, if any one anionic surfactant is present in the wash composition, the anionic surfactant is present at less than 2 wt. % in the composition. For example, the amount of anionic surfactant is less than 1 wt. %.

Various non-ionic, cationic, amphoteric or zwitterionic surfactants suitable for use in the invention are disclosed in McCutcheon's Detergents and Emulsifiers, North American Edition, McCutcheon Division, MC Publishing Co., Glen Rock, N.J. 07452 and the CTFA International Cosmetic Ingredient Handbook, Published by The Cosmetic, Toiletry, and Fragrance Association, Washington, D.C.

One particular class of non-ionic surfactants that can be used in the wash compositions are the polyether surfactants that comprise one or more chains or polymeric components having oxyalkylene (—O—R—) repeats units wherein R has 2 to 6 carbon atoms. Representative polyether surfactants comprise block polymers of two or more different kinds of oxyalkylene repeat units, the ratio of which determining the HLB of the surfactant. The chain polyether surfactants employed in the invention can be selected, for example, from the group of commercially available surfactants having the name poloxamine or poloxamer, as adopted by The CTFA International Cosmetic Ingredient Dictionary. These class of surfactants are commercially available from BASF, and are often referred to by their tradenames Tetronics® and Pluronics®.

Tetronics® and Pluronics®. are block copolymers consisting of propylene oxide (PO) and ethylene oxide (EO) blocks—specifically, they are poly(a-oxyethylene-b-oxypropylene-a-oxyethylene) triblock copolymers. Their solubility in water is generally good, but the properties of the individual block copolymers vary substantially. The nomenclature used for the block copolymers, and generally herein, is such that the first two figures, when multiplied by 100, represent the average molecular weight of the PO block, whilst the last figure, when multiplied by 10, represents the ethylene oxide content (% w/w) of the poloxamer. Thus, for Pluronic® F127, the average molecular weight of the PO block is 12000 Daltons with 70% w/w/ethylene oxide content.

Another class of non-ionic surfactants include cocamide MEA and oleaimide MEA.

The wash compositions can also include an amphoteric surfactant, which can provide thickening and high foaming properties to the composition. The preferred classes of amphoteric surfactant are sultaines and betaines. Preferably, the amphoteric surfactant is a sultaine. More preferably, the amphoteric surfactant is a mixture of a sultaine and a betaine.

The preferred sultaine is cocamidopropyl hydroxysultaine. The preferred betaine is cocamidopropyl betaine. Preferably, the total amount of amphoteric surfactant is from 1 wt. % to 5 wt. % of the total weight of the wash composition. More preferably, the total amount of amphoteric surfactant is from 1.5 wt. % to 3 wt. % of the total weight of the composition. In one preferred embodiment, the amphoteric surfactant is from 0.5 wt. % to 1.5 wt. % sultaine, and from 0.5 wt. % to 1.5 wt. % betaine.

The wash compositions can also include a zwitterionic surfactant. Zwitterionic surfactants are broadly described as derivatives of aliphatic quaternary ammonium, phosphonium, and sulfonium compounds, in which the aliphatic radicals can be straight or branched chain, and wherein one of the aliphatic substituents contains from about 8 to about 18 carbon atoms and one contains an anionic group, e.g., carboxy, sulfonate, sulfate, phosphate, or phosphonate.

Examples of zwitterionic surfactants include: 4-[N,N-di(2-hydroxyethyl)-N-octadecylammonio]-butane-1-carboxylate; 5-[S-3-hydroxypropyl-S-hexadecylsulfonio]-3-hydroxypentane-1-sulfate; 3-[P,P-diethyl-P-3,6,9-trioxatetradexocylphosphonio]-2-hydroxypropane-1-phosphate; 3-[N,N-dipropyl-N-3-dodecoxy-2-hydroxypropylammonio]-propane-1-phosphonate;3-(N,N-dimethyl-N-hexadecylammonio)propane-1-sulfonate; 3-(N,N-dimethyl-N-hexadecylammonio)-2-hydroxypropane-1-sulfonate; 4-[N,N-di(2-hydroxyethyl)-N-(2-hydroxydodecyl)ammonio]-butane-1-carboxylate; 3-[S-ethyl-S-(3-dodecoxy-2-hydroxypropyl)sulfonio]-propane-1-phosphate; 3-[P,P-dimethyl-P-dodecylphosphonio]-propane-1-phosphonate; and 5- [N,N-di(3-hydroxypropyl)-N-hexadecylammonio]-2-hydroxy-pentane-1-sulfate.

The wash composition will also include an antimicrobial agent that is compatible with ocular tissue. Accordingly, the compositions include one or more antimicrobial agents common to many multipurpose contact lens solutions including, but are not limited to, 1,1′-hexamethylene-bis[5-(p-chlorophenyl)biguanide](chlorhexidine) or water soluble salts thereof, 1,1′-hexamethylene-bis[5-(2-ethylhexyl)biguanide](alexidine) or water soluble salts thereof, poly(hexamethylene biguanide) (PHMB) or water soluble salts thereof, α-[4-tris(2-hydroxyethyl)ammonium chloride-2-butenyl]poly[1-dimethylammonium chloride-2-butenyl]-ω-tris(2-hydroxyethyl)ammonium chloride (available as polyquaternium-1 from Stepan Corporation), cetyl pyridinium chloride and quaternary ammonium esters. The preferred antimicrobial agents are PHMB, and most preferably, the antimicrobial agent is alexidine. In another embodiment, the antimicrobial agent is polyquaternium-1.

The antimicrobial agent can also be a fatty acid monoester. The fatty acid monoester comprises an aliphatic fatty acid portion having six to fourteen carbon atoms and an aliphatic hydroxyl portion. The term “aliphatic” refers to a straight or branched, saturated or unsaturated hydrocarbon. The aliphatic hydroxyl portion of the fatty acid monoester can be any aliphatic compound with at least one hydroxyl group, preferably at least two hydroxyl groups. In many of the embodiments, the aliphatic hydroxyl portion will have from three to nine carbons. The aliphatic hydroxyl portion includes, but is not limited to, propylene glycol, glycerol, a polyalkylene glycol, e.g., polyethylene glycol or polypropylene glycol, a cyclic polyol, e.g., sorbitan, glucose, mannose, sucrose, fructose, fucose and inisitol and derivatives thereof, and a linear polyol, e.g., mannitol and sorbitol and derivatives thereof. In a preferred embodiment the aliphatic hydroxyl portion is glycerol.

In one embodiment, the aliphatic fatty acid portion is a straight chain, saturated or unsaturated hydrocarbon with eight to ten carbons, e.g., decanoylglycerol. In another embodiment, the aliphatic fatty acid portion is a branched chain, saturated or unsaturated hydrocarbon with eight to ten carbons. For example, a 5-ethyl substituted derivative of decanoylglycerol can be used. Moreover, if the fatty acid portion is unsaturated, it is preferred that the fatty acid is monounsaturated, with the cis-form being preferred over the trans-form.

The wash composition can also include a moisturizing agent. Typically the moisturizing agent is polyethylene glycol, methylcellulose, hydroxypropylmethylcellulose, povidone, polyquaternium-10, polyquaternium-24, polyquaternium-67. The moisturizing agent is present in an amount that is a minimum of 0.02 wt. % and a maximum of 8.0 wt. %. In one embodiment, the moisturizing agent is preferably a cationic moisturizing agent.

In one embodiment, the moisturizing agent is a cationic polysaccharide. Examples of cationic polysaccharides include but are not limited to polyquaternium-10, polyquaternium-24, polyquaternium-67. Preferably, in one embodiment, the cationic polysaccharide is polyquaternium-10 available from Dow Chemical Company, Midland, Mich. In one embodiment, the cationic moisturizing agent, polymeric cationic moisturizing agent and/or cationic polysaccharide, including but not limited to polyquaternium-10, are present in an amount that is a minimum of 0.01 wt. %, 0.05 wt. % or 0.1 wt. % and/or a maximum of 8 wt. %, 5 wt. %, 1 wt. % or 0.5 wt. %. In one preferred embodiment, the wash composition includes polyquaternium-10 which is present from 0.05 wt. % to 0.5 wt. %

In another embodiment, the moisturizing agent is a non-polymeric cationic moisturizing agent. The non-polymeric cationic moisturizing agent is a cationic saccharide. In one preferred embodiment, the non-polymeric cationic moisturizing agent is coco monoethanolamide (Cocoamide, Pfaltz & Bauer, Waterbury, Conn.) ethoxylated methyl glucose ether (Glucam E-20, Dow Chemical, Midland, Mich.) or decyl glucoside. In one preferred embodiment, the amount of non-polymeric cationic moisturizing agent is a minimum of 0.01 wt. %, 0.05 wt. %, 0.1 wt. %, 0.5 wt. % or 1 wt. % and/or a maximum of 8 wt. % or 5 wt. %.

Alternatively, the non-polymeric cationic moisturizing agent is a polyol. Typically, the polyol is selected from the group consisting of glycerin, propylene glycol, and sugar alcohols. In one preferred embodiment, the polyol is glycerin or propylene glycol. In another preferred embodiment, the amount of polyol agent is a minimum of 0.01 wt. %, 0.05 wt. %, 0.1 wt. %, 0.5 wt. % or 1 wt. % and/or a maximum of 8 wt. %, 5 wt. %, 2 wt. % or 1 wt. %.

The wash compositions can also include a branched, glycerol monoalkyl compound is selected from a branched, glycerol monoalkyl ether, a branched, glycerol monoalkyl amine or a branched, glycerol monoalkyl sulfide or any mixture thereof. For example, each of the respective monoalkyl ether, monoalkyl amine and monoalkyl sulfide can be 3-[(2-ethylhexyl)oxy]-1,2-propanediol (EHOPD), 3-[(2-ethylhexyl)amino]-1,2-propanediol (EHAPD), 3-[(2-ethylhexyl)thiol]-1,2-propanediol (EHSPD) or any mixture thereof. The chemical structures of EHOPD, EHAPD and EHSPD are provided below.

EHOPD is also referred to as octoxyglycerin as is sold under the tradename Sensiva® SC50 (Schülke & Mayr). EHOPD is a glycerol alkyl ether known to be gentle to the skin, and to exhibit antimicrobial activity against a variety of Gram-positive bacteria such as Micrococcus luteus, Corynebacterium aquaticum, Corynebacterium flavescens, Corynebacterium callunae, and Corynebacterium nephredi. Accordingly, EHOPD is used in various skin deodorant preparations at concentrations between about 0.2 and 3 percent by weight. EHAPD can be prepared from ethylhexylamine and 2,3-epoxy-1-propanediol using chemistry well known to those of ordinary skill in the art. EHSPD can be prepared from 2-ethylhexylthiol and 2,3-epoxy-1-propanediol using chemistry well known to those of ordinary skill in the art.

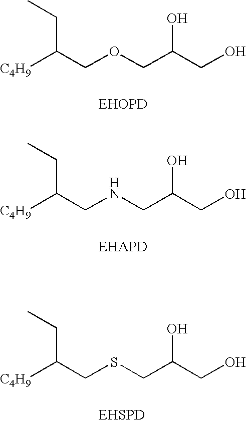

The wash composition can also include one or more conventional buffers to obtain and maintain a desired pH range of the composition. Suitable buffers include for example but are not limited to borate buffers based on boric acid and/or sodium borate, phosphate buffers based on Na2HPO4, NaH2PO4 and/or KH2PO4, citrate buffers based on sodium or potassium citrate and/or citric acid, sodium bicarbonate, aminoalcohol buffers, Good buffers and combinations thereof. Generally, buffers will be used in a minimum amount of about 0.05 wt. % or about 0.1 wt. % and a maximum amount of about 1.5 wt. % or about 2.5 wt. %.

According to one embodiment, the wash composition is formulated according to the following compounds and concentrations of Table 1.

According to one embodiment, the wash composition is formulated according to the following compounds and concentrations of Table 2.

According to one embodiment, the wash composition is formulated according to the following compounds and concentrations of Table 3.

TABLE 1

| | Minimum | Maximum |
|---|---|---|
| Compound | (wt. %) | (wt. %) |
| pluronic ® F127 | 10 | 30 |
| propylene glycol | 0.5 | 3 |
| Glucamate LT | 0.1 | 2 |
| decyl glucoside | 0.5 | 10 |
| citric acid | adj. to pH 5.5 | adj. to pH 7.5 |
| PHMB | 100 ppm | 0.001 |
| water | q.s. to 100% | q.s. to 100% |

TABLE 2

| | Minimum | Maximum |
|---|---|---|
| Compound | (wt. %) | (wt. %) |
| tetronic ® 1304 | 10 | 30 |
| Glucam E-20 | 0.5 | 10 |
| glycerin | 0.5 | 5 |
| citric acid | adj. to pH 5.5 | adj. to pH 7.5 |
| alexidine HCl | 100 ppm | 0.005 |
| water | q.s. to 100% | q.s. to 100% |

TABLE 3

| | Minimum | Maximum |
|---|---|---|
| Compound | (wt. %) | (wt. %) |
| pluronics ® F68 | 10 | 30 |
| decyl glucoside | 0.5 | 10 |
| cocamide MEA | 0.5 | 10 |
| HPMC | 0.05 | 1 |
| citric acid | adj. to pH 5.5 | adj. to pH 7.5 |
| polyquarternium-1 | 10 ppm | 500 ppm |
| ethylhexyl glycerol | 0.01 | 1 |
| water | q.s. to 100% | q.s to 100% |

The wash composition can be made available in individual packets, pump dispenser or squeeze bottles. In another embodiment, the wash composition can be sold as a kit with a multipurpose contact lens cleaning solution. The kit combines the convenience of having a wash composition packaged with a compatible multipurpose solution. As a result, the kit greatly assists contact lens wearers with the recommended maintenance of their lenses, but more importantly, the kit assists the lens wearers to minimize lens contamination that can occur through handling of the lens with contaminated hands or fingers. Accordingly, the kit includes a wash composition comprising one or more surfactants selected from the group consisting non-ionic, cationic, amphoteric or zwitterionic surfactants, and a multipurpose contact lens solution.

In particular, the wash composition is suitable for use with the multipurpose contact lens solution such that any one component in the wash composition at a reduced concentration of 5% the concentration in the wash composition does not negatively impact the biocidal efficacy of the multipurpose solution. For example, let us assume that component A in the wash composition is shown to have some negative impact on the biocidal efficacy of the multipurpose solution. To determine whether component A can be used in the wash composition, one would determine if component A still has a statistically significant negative impact on biocidal efficacy though at a concentration that is 5% the concentration of component A in the wash composition. Biocidal efficacy is determined by protocols well known by those of ordinary skill ion the art according to the Stand-alone Biocidal procedure outlined in ISO 14729, International Standardized Document for Ophthalmic Optics and FDA Premarket Notification (510 k) Guidance Document for Contact Lens Care Products.

In a preferred embodiment, any one component in the wash composition at a reduced concentration of 10% the concentration in the wash composition does not negatively impacts the biocidal efficacy of the multipurpose solution.

### Method of Use

The present composition is used in one embodiment by first rinsing hands, applying the wash composition, lathering the hands and rinsing the composition from the hands. Preferably, the wash composition is in the form of a liquid or gel. These steps may be repeated as desired by the user. The hands are optionally dried by hand towels, paper towels or air dryer. Following the washing of the hands the contact lenses can be handled, that is, the contact lenses can be removed from the eye for cleaning and/or storage or inserted into the eye from the lens package or the disinfecting lens case.

### Definitions

“Non-ionic” as it refers to a surfactant means any surfactant that does not possess an ionic charge on the surfactant.

“Anionic” as it refers to a surfactant means any surfactant that possesses an anionic charge and does not possess an additional cationic charge on the surfactant.

“Cationic” as it refers to a surfactant means any surfactant that possesses a cationic charge and does not possess an additional anionic charge on the surfactant.

“Polyol” means any alcohol that contains two or more hydroxyl groups.

### EXAMPLES

### Example 1

The compounds listed in formula A are combined to make a liquid hand cleaner to be used before handling contact lenses. The composition does not introduce any components that are expected to lower the antimicrobial efficacy of a contact lens cleaning solution.

Formula A: Hand Cleaner

| Compound | % w/w |
|---|---|
| pluronic F127 | 20.00 |
| propylene glycol | 1.00 |
| Glucamate LT | 0.50 |
| decyl glucoside | 3.00 |
| citric acid | pH - 5.5-7.5 |
| PHMB | 50 ppm |

### Example 2

The compounds listed in formula B are combined to make a liquid hand cleaner to be used before handling contact lenses. The composition does not introduce any components that are expected to lower the antimicrobial efficacy of a contact lens cleaning solution.

Formula B: Hand Cleaner

| Compound | % w/w |
|---|---|
| tetronic 1304 | 20.00 |
| Glucam E-20 | 2.00 |
| glycerin | 1.00 |
| citric acid | pH - 5.5-7.5 |
| alexidine | 50 ppm |

### Example 3

The compounds listed in formula C are combined to make a liquid hand cleaner to be used before handling contact lenses. The composition does not introduce any components that are expected to lower the antimicrobial efficacy of a contact lens cleaning solution.

Formula C: Hand Cleaner

| Compound | % w/w |
|---|---|
| pluronic F68 | 20.00 |
| decyl glucoside | 5.00 |
| cocamide MEA | 3.00 |
| Hydroxpropylmethylcellulose | 0.50 |
| polyquarternium-1 | 50 ppm |
| citric acid | pH - 5.5-7.5 |
| ethylhexyl glycerol | 0.20 |

What is claimed is:

1. A method for minimizing microbial contamination of contact lenses during the handling of the lenses, the method comprising:
instructing a person that wears contact lenses to wash their hands prior to the handling of the contact lenses with a wash composition comprising:
one or more surfactants selected from the group consisting of non-ionic, cationic, amphoteric or zwitterionic surfactants, and optionally, an anionic surfactant, wherein the anionic surfactant, if present, is present at a concentration that is 30 wt. % or less of the total surfactant concentration; and
an antimicrobial agent that is compatible with ocular tissue.

2. The method of claim 1 wherein the anionic surfactant, if present, is present at a concentration that is 10 wt. % or less of the total surfactant concentration.

3. The method of claim 1 to 2 wherein the concentration of anionic surfactant in the composition is less than 2 wt. %.

4. The method of claim 1 to 3 wherein the total surfactant concentration in the composition is from 5 wt. % to 40 wt. %.

5. The method of claim 1 to 4 wherein the composition further comprises a moisturizing agent.

6. The method of claim 1 to 5 wherein the composition further comprises a polyol.

7. The method of claim 1 to 6 wherein the antimicrobial agent is selected from the group consisting of poly(hexamethylene biguanide) and alexidine.

8. The method of claims 1 to 4 wherein the antimicrobial agent is selected from the group consisting of poly(hexamethylene biguanide), chlorhexidine, alexidine, polyquaternium-1, cetylpyridinium chloride, quaternary ammonium esters and fatty acid monoglycerides.

9. The method of claims 1 to 4 wherein the handling of the contact lenses includes the rubbing of the lenses to assist in the cleaning and disinfection of the lenses, or inserting the lenses in the eyes.

10. The method of claims 1 to 9 wherein the wash composition is in the form of a liquid or gel.

11. A kit for to assisting contact lens wearers with the care maintenance of their lenses, the kit comprising:
a wash composition comprising one or more surfactants selected from the group consisting of non-ionic, cationic, amphoteric or zwitterionic surfactants, and optionally, an anionic surfactant, wherein the anionic surfactant, if present, is present at a concentration that is 30 wt. % or less of the total surfactant concentration and an antimicrobial agent that is compatible with ocular tissue; and
a multipurpose contact lens solution, wherein the wash composition is suitable for use with the multipurpose contact lens solution such that any one component in the wash composition at a reduced concentration of 5% the concentration in the wash composition does not negatively impacts the biocidal efficacy of the multipurpose solution.

12. The kit of claim 11 wherein the any one component in the wash composition at a reduced concentration of 10% the concentration in the wash composition does not negatively impacts the biocidal efficacy of the multipurpose solution.

13. The kit of claim 11 and 12 wherein the antimicrobial agent is selected from the group consisting of poly(hexamethylene biguanide), chlorhexidine, alexidine, polyquaternium-1, cetylpyridinium chloride, quaternary ammonium esters and fatty acid monoglycerides.

14. The kit of claims 11 and 12 wherein the antimicrobial agent is selected from the group consisting of poly(hexamethylene biguanide) and alexidine.
